(19) United States
(12) Patent Application Publication
(10) Pub. No.: US 2026/0224823 A1
(43) Pub. Date: Aug. 6, 2026

(54) GAS CONTROL IN MEDICAL FLUID FILTERS

(71) Applicant: TESSEN SOLUTIONS LIMITED, Carrigaline (IE)

(72) Inventors: Vincent FORDE, Carrigaline (IE); Pauric CAREY, Carrigaline (IE); James ROCHE, Carrigaline (IE)

(73) Assignee: TESSEN SOLUTIONS LIMITED, Carrigaline (IE)

(21) Appl. No.: 19/151,281

(22) PCT Filed: Jan. 25, 2024
(86) PCT No.: PCT/EP2024/051816

§ 371 Date: Jul. 25, 2025

(30) Foreign Application Priority Data
Jan. 27, 2023 (EP) 23153817.4

(51) Int. Cl. A61M 5/38; A61M 5/14; A61M 39/24

(52) U.S. Cl. CPC A61M 5/385; A61M 39/24; A61M 2005/1402; A61M 2039/242

(57) ABSTRACT

Gas trap and particle filter devices for uses such as intravenous delivery are described. They effectively prevent gas bubbles from migrating distally towards a patient in use. They may have a priming port (7) which is used only once and permanently sealed at priming. This port has a backcheck valve (10) which allows escape of air during priming but prevents air from entering the chamber. Such a valve is described both proximally of, and at, a particle filter (230 ) in other devices, contributing to very effective filtration with minimum risk of gas flowing onward to a patient.

### INTRODUCTION

The present invention relates to medical or veterinary fluid filters including gas traps and particulate filters.

When administering hazardous drugs, biohazardous fluids or other fluids it is important to maintain a closed system that eliminates ingress of contaminants and egress of toxic liquids, gases or vapours. In practice, a closed system is preferred end-to-end to increase safety for both the patient and the healthcare practitioner. A closed system infers that the drug is delivered directly into the patient's venous system without opening the IV system that carries the fluids from the reservoir, typically a bag or bottle, through a tube system and venous entry device, such as a catheter or needle. In practice there may be one or several devices attached to the line, for example, to allow access where additional drugs are required. There are a variety of components that enable access to the IV system, such as stopcocks, multi-way taps, y-connectors, Luer connectors or other IV components intended to be accessed by a third-party device. Each time a break is made in the line, or a component attached or accessed, there is an increased risk of pressure drop that can result in air being pulled into the IV line. For example, a standard Luer or push connection may be erroneously left open or under-tightened so as to not create an air-tight seal. It is therefore a recognised preference among healthcare practitioners to reduce or eliminate the number of connection devices or manual manipulations of an IV. Furthermore, the liquid drugs being delivered may be volatile or chemically unstable, resulting in degassing of the liquids.

Sublimation, the transition of solid substance to a gas, can occur spontaneously in certain drugs. Another source of air in IV lines comes from temperature changes in the liquid as they are administered over a period of time. Many fluids, such as blood and other drugs, are stored in refrigerators as best practice, these drugs are then left to warm to ambient temperatures or actively warmed to body temperature to reduce hypothermic reactions in patients. For chemotherapy or other hazardous drugs, compounding activities are typically done in the Pharmacy in hospitals, under controlled conditions that reduce risk of exposure of the drug vapours or spills of the liquid, which may be hazardous to human health. Exposure of hazardous drugs is a recognised hazard that results in increased rates of infertility, reproductive issues and cancers among healthcare workers.

When administering chemotherapy or other hazardous drugs, nurses will employ a variety of methods to set up the IV system. The drug may be given by gravity induced flow or by IV pump. The drug may be given as a single dose or combined with other fluids, a setup typically known as piggyback or secondary infusion. The setup of the giving set can vary depending on drugs being used, hospital policy, economic capacity of the facility or any other reasons. In any IV infusion, a closed system is preferred to protect both healthcare worker and patient.

For chemotherapy administration, a typical scenario is for the drug to be compounded in the pharmacy and delivered to the oncology department, where administration is handled by nurse professionals. These nurses will often wear appropriate PPE (Personal Protective Equipment) and employ a sterile process while hanging the IV fluids. In some scenarios a patient may be receiving several fluids in a single chemotherapy sitting and they may already have an active infusion underway. Typical chemotherapy sittings can range from one to more than one drug, as well as non-hazardous drugs such as saline solution, being administered for up to or more than one hour at a time.

Air-in-Line is the commonly used term to describe bubbles of air that are present in the IV tube during administration, these bubbles being recognised as a hazard due to the risk of air embolism. Vascular Air Embolism (VAE) is the result of any quantity of air entering the venous system. Air is foreign to the venous system and can trigger a number of adverse reactions, such as inflammation, destruction of endothelial cells, inducement of the coagulation cascade, blockage of blood flow to critical organs or tissue, heart complications. The most serious events occur when air enters the arterial blood supply, which can result in migration of micro-bubbles into cortical tissue, resulting in ischemic strokes and cognitive dysfunction in patients. Patients with a patent foramen ovalae, commonly known as hole in the heart, a condition which is believed to affect up to 40% of the adult population and all infants, are at much higher risk of air migrating into the left side of the heart and passing freely to the brain. This is known as a paradoxical air embolism and can result in catastrophic damage to brain function with even tiny amounts of air. For these reasons, a high degree of safety is practiced in healthcare to ensure air is not allowed to pass into the patient's vasculature.

Another aspect of drug delivery directly into the patient is risk from solid particles causing a thrombus, where a solid object blocks blood flow in the venous system. These particles are foreign objects in the body that lead to a coagulation cascade and inflammation response as the body tries to expel the foreign object. Large particles are at risk of reaching end-arterial locations and cutting off blood supply to critical tissue or organs. In certain situations, this can cause tissue damage, necrosis, heart arrythmia and brain injury. Drugs can precipitate to form crystals due to environmental changes or as they chemically combine in IV infusion systems, with certain drugs known to be particularly risky for particle formation. In the manufacturing process micro plastic and glass particles may be retained in the bottles, bags, tubes and components of the IV system. These micro-particles must be prevented from entering the body similarly. In clinical practice it is common to use particle filters in IV infusion systems.

For many critical drugs that have a short half-life, such as chemotherapy, or where accuracy of flow rate is important, active devices such as IV pumps are used to control flow. These pumps may be activated by syringe or peristaltic of other method to pull a specified volume of the fluids form the reservoir to the patient. The IV pumps usually include a sensor device to identify when Air-in-line is present and a warning system to alert medical professionals to the danger, the pump then stopping the IV infusion until the air is cleared. Typically, a nurse must manually intervene to remove the bubble, often using a variety of methods including tapping, flicking or agitating the line, or attaching a third-party device to suck the air from the line. The method of removing the AIL can depend on the IV setup.

Various devices are disclosed in our published specification numbers WO2020/156666 and WO2022/023079. Such devices include a diverter between chamber inlet and outlet ports, and a diffuser in the inlet with holes in a radial arrangement.

During intravenous (IV) infusion natural degassing can occur. In one example, this can occur if there are two or more chemically incompatible fluids, leading to bubbles of gas that can pose a threat to the health of a patient.

In-line filters are frequently used in medical settings and are indicated for use with some drugs that are known to precipitate, trapping the solid particles in a membrane, especially required in the case of premature or paediatric patients whose vasculature cannot withstand the burden of air or solid particles entering the body. Open in-line filters have known complications that can lead to clinical risk caused by blood backing up or back-siphoning, which can be caused by not closing the clamp below the filter to maintain the prime in the device or placing the open in-line filter higher than the level of the patient causing a loss of prime. In the latter case, subsequently lowering the unprimed device below the level of the patient's mid-axillary level or IV site during an infusion can then lead to back-siphoning. In one scenario the patient may receive an overdose of drug, leading to harm, or in the other scenario an underdose of drug, leading to poor treatment and slower recovery. In the clinical setting if improperly placed an open filter can cause blood to back siphon, leading to blood loss and potential injury. These limitations make the open in-line filters a potential cause of harm if improperly handled or moved during the infusion. In the case of open filters, the drugs passing through the device are vented to atmosphere with risk of exposure of toxic and harmful drug gases such as chemotherapy. It can be appreciated that a closed in-line filter may not present these handling challenges or inherent risks.

The present invention is directed towards providing enhanced trapping of gas and/or particles before it flows onward to a patient.

### SUMMARY

We describe a fluid filter device for blocking of gas and/or particles in a medical fluid line, the device comprising an inlet, an outlet, and a flow chamber between said inlet and said outlet for flow in a flow direction distally from the inlet to the outlet, the flow chamber having an active component for trapping gas and/or particles, and the chamber having a port with a backcheck valve to allow escape of gas through said port but not inflow of gas through said port. The term backcheck valve means a valve which allows flow in one direction only.

In some preferred examples, the flow chamber comprises a gas trap, with the outlet configured for flow of only liquid with gas being trapped in the chamber either temporarily or permanently.

In some preferred examples, the device comprises a priming port for outflow of air from the flow chamber during priming and sealing to close the chamber for use.

In some preferred examples, the backcheck valve is part of the priming port. In some preferred examples, there is a backcheck valve which is separate from the priming port.

In some preferred examples, the backcheck valve comprises a pair of leaflets which are normally converged together and separate under air flow pressure from within the chamber.

In some preferred examples, the flow chamber houses a particle filter.

In some preferred examples, the flow chamber comprises a port with a backcheck valve proximally In some preferred examples, the flow chamber comprises a port with a backcheck valve located adjacent the filter to accept outflow of gas laterally from the filter and the flow direction. In some preferred examples, the device comprises an outer chamber for receiving gas which has flowed out through the port.

In some preferred examples, the outer chamber is in fluid communication with a plurality of ports on the flow chamber. In some preferred examples, the device comprises a pressure regulator port, comprising a backcheck valve linked with a volume formed by a flexible diaphragm.

In some preferred examples, the diaphragm forms a volume to trap gas which flows through the port, thereby preventing any toxic gases from escaping to the surrounding environment.

In some preferred examples, the port comprises a mesh to retard outflow of liquid. In some preferred examples, the mesh is hydrophobic.

In some preferred examples, the device comprises a particle filter and there is a backcheck valve in the outlet.

In some preferred examples, the device comprises a gas trap and a priming port including a backcheck valve for permitting only outflow of gas.

In some preferred examples, the device comprises a port with a closure such as a cap, and a backcheck valve included in said port.

We also describe a medical intravenous giving set comprising a fluid supply, a supply line, and a device of any example described herein connected in the line to trap gas to prevent it from flowing towards a patient.

### DETAILED DESCRIPTION OF THE INVENTION

The invention will be more clearly understood from the following description of some embodiments thereof, given by way of example only with reference to the accompanying drawings in which:

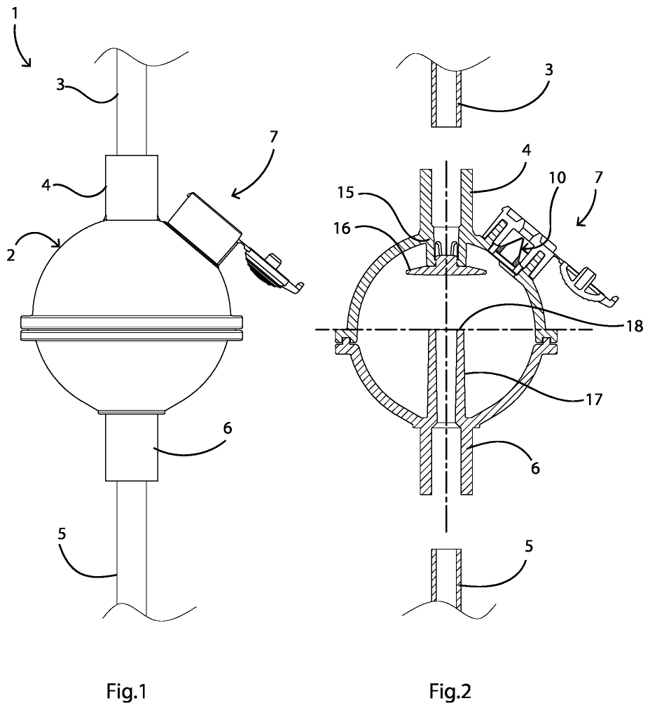

FIG. 1 is a front view of a gas trap device of the invention, and FIG. 2 is a front sectional view of the device, showing internal components,

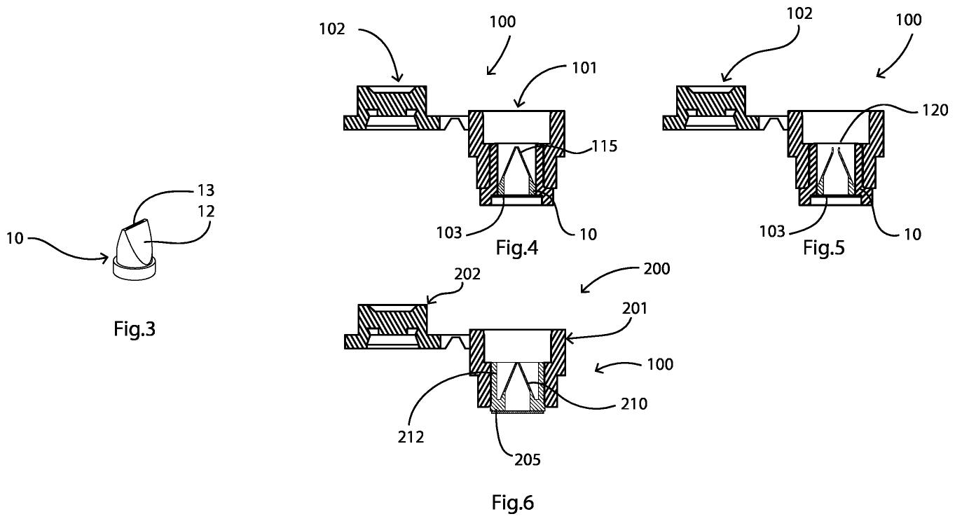

FIG. 3 is a perspective view of a backcheck valve of the device of FIGS. 1 and 2,

FIGS. 4 and 5 are front sectional views of a priming port, open and closed respectively, incorporating the backcheck valve and being suitable for use with a gas trap device or a particle filter device,

FIG. 6 is a front sectional view of an alternative priming port with a backcheck valve,

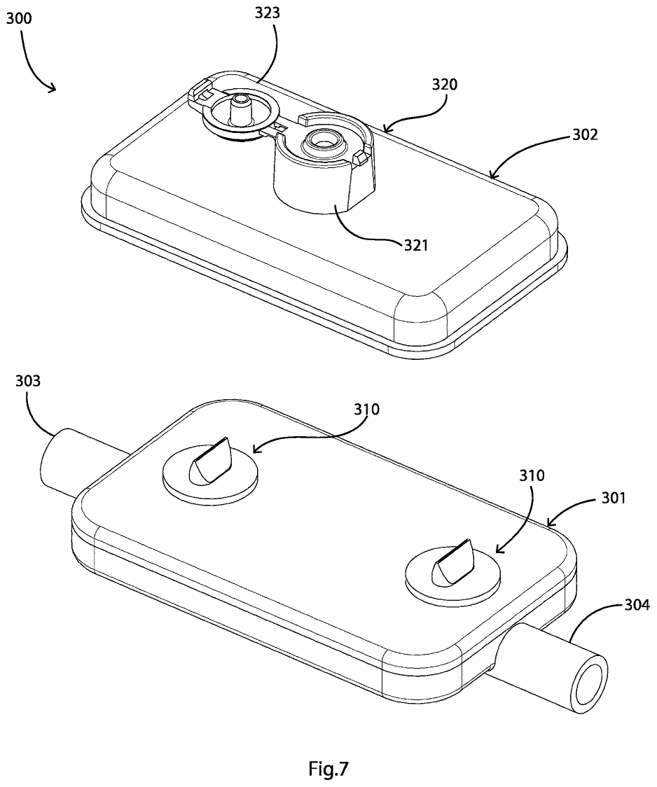

FIG. 7 is an exploded view of the major parts of a filter device of the invention, and:

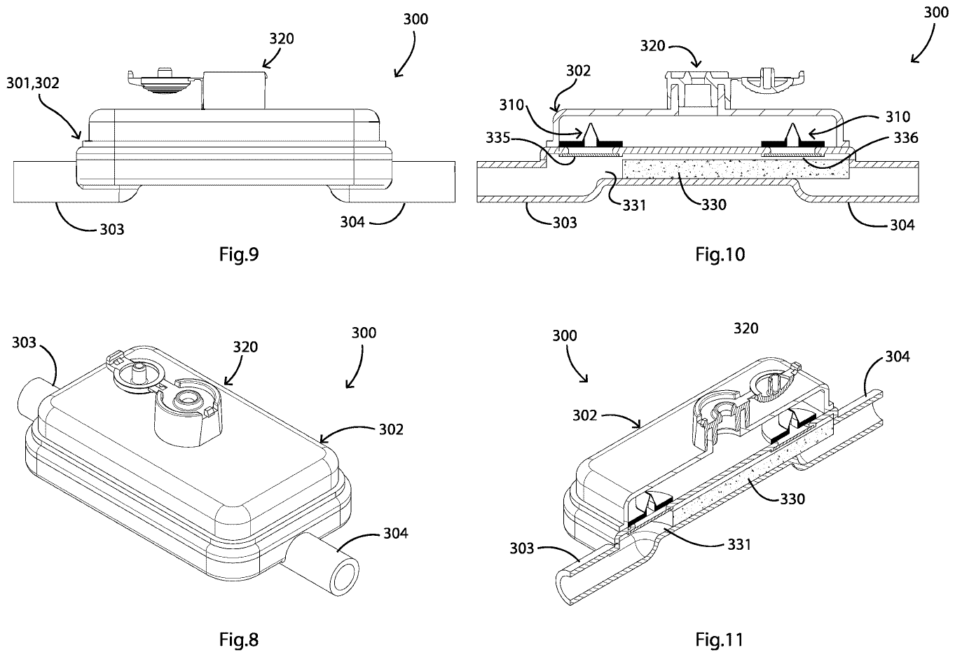

FIG. 8 is a perspective view of this device,

FIG. 9 is a front view of this device,

FIG. 10 is a front sectional view of this device showing internal components, and

FIG. 11 is a perspective cut-away view showing this device,

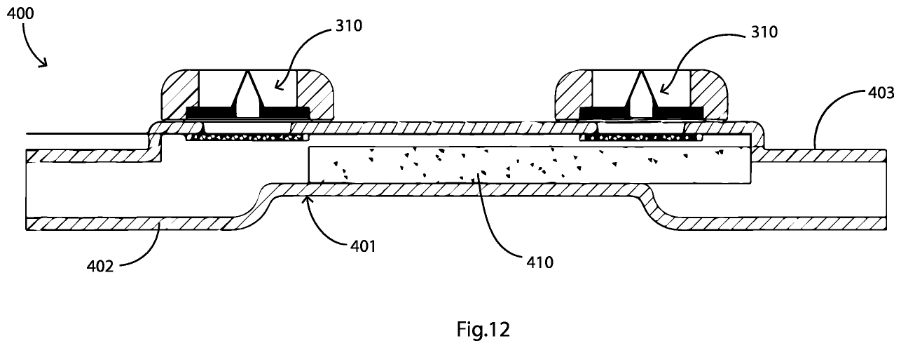

FIG. 12 is a front sectional view of another particle filter of the invention,

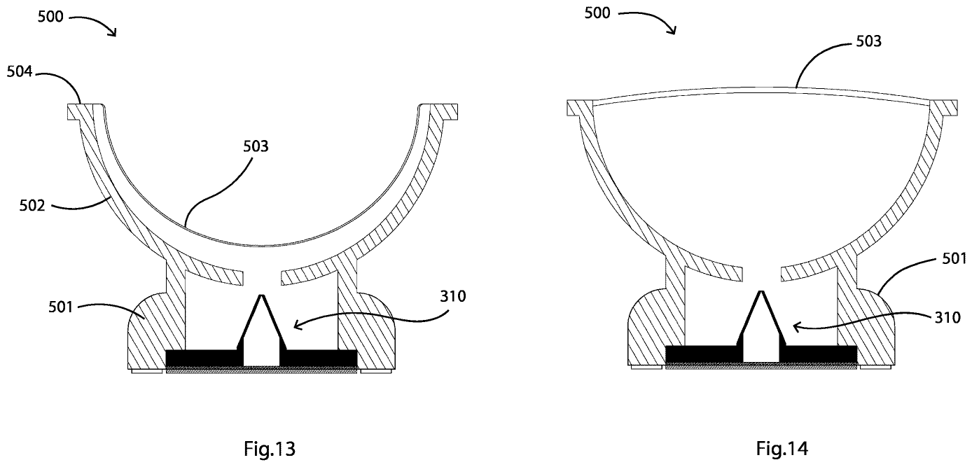

FIGS. 13 and 14 are front sectional views of a pressure regulating port for a gas trap or particle filter device of the invention, before and after use respectively,

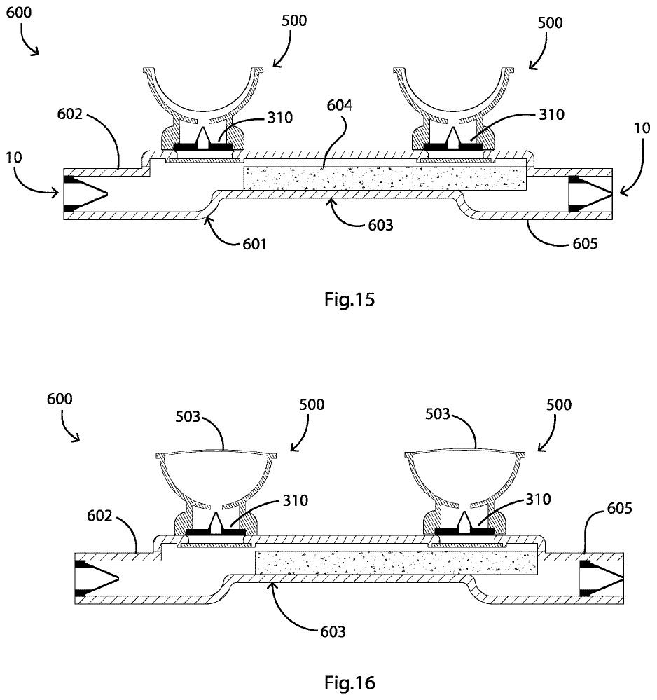

FIGS. 15 and 16 are front sectional views showing a particle filter before and after use of pressure regulating ports, respectively,

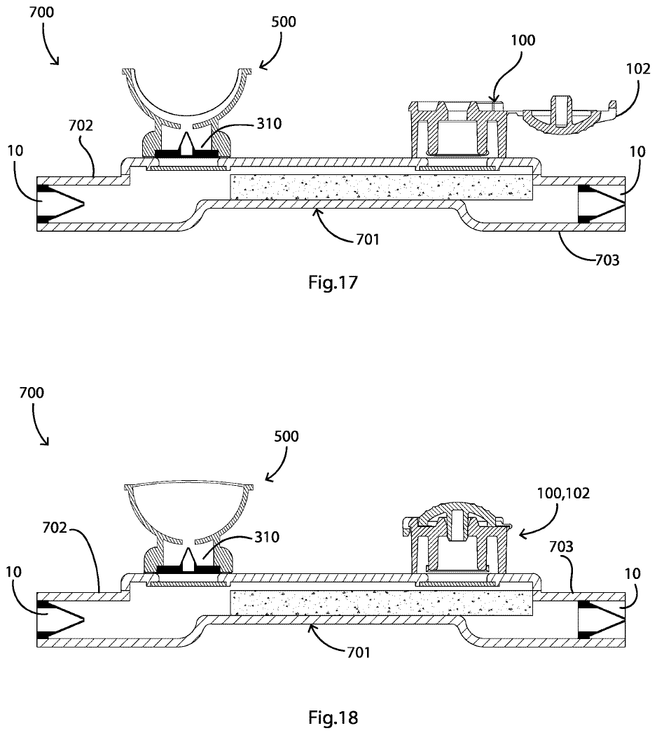

FIGS. 17 and 18 are front sectional views of another particle filter device, in this case having a pressure regulator valve and a priming port,

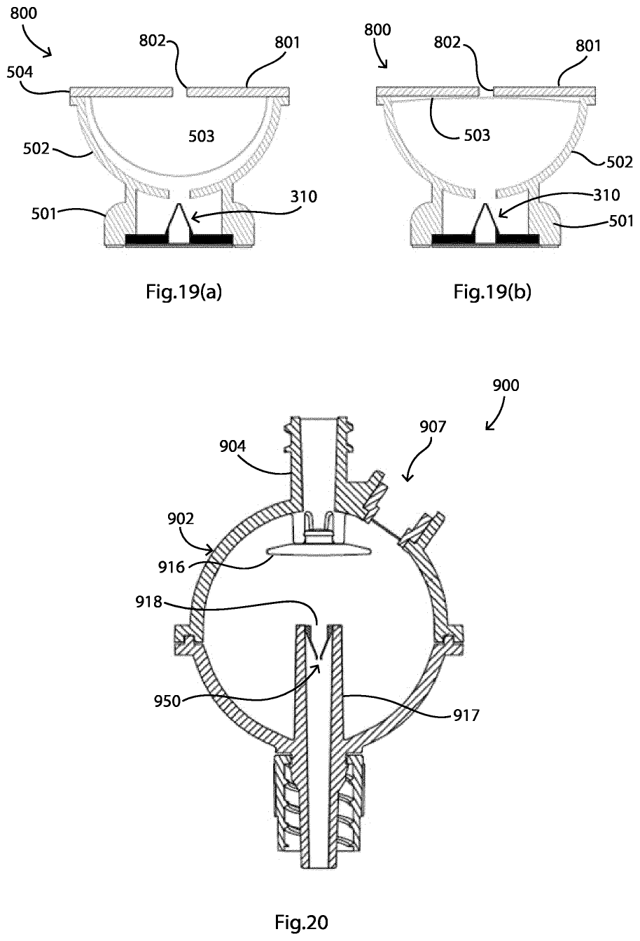

FIG. 19(a) and (b) are front sectional views showing a pressure regulating port for a gas trap or a particle filter, in this case the port having a rigid cover, and

FIG. 20 is a front cross-sectional view of a gas trap device having a backcheck valve in its outlet conduit.

### DETAILED DESCRIPTION OF THE EMBODIMENTS

Referring to FIGS. 1 to 3 a gas trap device 1 has a spherical chamber 2, linked to inlet and outlet flow lines 3 and 5 by inlet and outlet ports 4 and 6 respectively. There is a priming port 7. Inlet flow enters via the inlet port 4 and flows radially out via diffuser openings 15. This flow is guided by a diverter 16, which has a proximal (closest to the inlet) surface which is tapered radially and distally around its rim. The combined effect of the diffuser openings 15 and the diverter 16 are that there is an initial turbulent region proximally of the diverter 16 which has the effect of separating in the form of bubbles any gas from the inlet fluid and such gas remains in the top (proximal region) of the chamber 2. Liquid flows radially guided by the proximal surface of the diverter 16, in a laminar manner. This liquid then migrates within the chamber through an outlet opening 18 and distally flowing within an outlet conduit 17 and out through the outlet port 6 to the outlet line 5.

The priming port 7 is used for initial priming of the line. Liquid is directed into the chamber 2 either flowing distally via the port 4 or proximally via the port 5, and it fills the chamber 2 until all air is expelled via the open priming port 7. Then, the priming port 7 is sealed by non-reversible closure of a cap. Moreover, outflow of air is one-way due to presence of a backcheck valve 10 within the priming port 7. This comprises a base supporting two valving flexible walls 12 and 13, which are biased to a converged closed configuration. Upon pressure being applied radially outwardly by air being purged during priming the walls 12 and 13 separate sufficiently for the outflow to continue, but in absence of such pressure they close together to prevent inflow of air. The priming port may take different forms. FIGS. 4 and 5 show a priming port 100 for use with a gas trap device or with a particle filter device. The priming port 100 comprises a main body 101 and a hinged cap 102, and the body 101 houses the backcheck valve 10. The valve body 101 is configured to be press fitted within a rim or sleeve of the chamber body. In addition, the port 100 comprises a disc-shaped filter 103 across its base. This filter is charcoal or a mesh having a hydrophobic coating to allow passage of air but to prevent flow of a liquid such as saline during priming.

FIG. 6 shows an alternative priming port, 200, in this case having a port body 201 and a hinged cap 202. In this case the backcheck valve, 210, has a tube 212 within which the pair of resilient leaves or walls are housed. Again, there is a base mesh filter, 205, with a hydrophobic coating.

As noted above backcheck valves may be used advantageously with fluid flow devices other than gas traps, such as particle filters. FIGS. 7 to 11 show a particle filter 300 having a main housing part 301 over which there is a cover housing part 302. The main housing part 301 has an inlet 303 and an outlet 304, and the top housing part 302 has a priming port 320 with a main body 321 and a hinged cap 323 for non-reversible closure of the port.

As shown particularly in FIGS. 10 and 11 the housing part 301 forms a flow chamber 331 housing a rectangular sheet particle filter 330. All inflowing fluid enters via the inlet 303 and flows through the filter 330 and out the outlet 304. Any gas which is present in the inlet fluid can flow outwardly through a first valve 310 and a second valve 310 located proximally of the filter 330 and over the filter 330. The first valve 310 has a hydrophobic mesh 335 on which resides a backcheck valve and the second valve has a hydrophobic mesh 336 over which there is also a backcheck valve 310. These valves allow outflow of gas only into a chamber formed by the cover housing part 302. This may be primed via the priming port, which is irreversibly sealed by a cap.

FIG. 12 shows a particle filter device 400, having a housing 401 forming an inlet 402 and an outlet 403 and a chamber in-between housing a particle filter 410. There are first and second valves 310, each over a hydrophobic membrane, in a top wall of the chamber 401, respectively proximally of the filter 410 and over the filter 410.

FIGS. 13 and 14 show a pressure regulator valve 500 comprising a base 501 housing a backcheck valve 310, and in fluid communication with a hemisphere 502 with a diaphragm 503. As gas flows through the valves 310 it fills the space under the diaphragm 503, so that pressure is regulated. Such pressure regulators may advantageously be used for particle filter devices. For example, FIGS. 15 and 16 show a device 600 with a housing 601 forming an inlet 602 and an outlet 605 and forming a chamber 603 in-between. The chamber 603 houses a particle filter 604. There is a valve 500 proximally of the filter 604 and one over the filter 604. Also, there is a backcheck valve 10 within each of the inlet 602 and the outlet 605 to provide for unidirectional fluid flow. FIG. 15 shows the device before use and FIG. 16 shows it after air has entered the space below the diaphragm 503.

The valve 500 may be used to assist in control of pressure within a flow conduit and chamber thereby helping to achieve consistency in flow of liquid to a patient. Also, advantageously, the valve 500 serves to prevent escaping gas from entering the surrounding environment, which is very beneficial if toxic drugs are being administered.

FIG. 17 and FIG. 18 show a device 700 having a housing 701 with an inlet 702 and an outlet 703 with a chamber in-between housing a particle filter. There is a pressure regulator 500 in the chamber proximally of the filter and a priming port 100 over the filter. Again, there are backcheck valves 10 for providing unidirectional flow through the device.

FIG. 19(a) and (b) show a pressure regulator valve 800 and parts similar to the valves of FIGS. 13 and 14 are given the same reference numerals. In this case there is additionally a rigid cover 801 over the diaphragm 503 with a hole 802 to allow air ingress for diaphragm movement. The cover 801 helps to protect the diaphragm from damage and from being pressed in. Such a valve may be used with either a gas trap device or with a particle filter device or with a combined device.

Referring to FIG. 20 a gas trap device 900 has a spherical chamber 901, an inlet port 904, a housing 907 for a priming port, a diverter 916, and an outlet conduit with an inlet opening 918 within the chamber 902. Function of the gas trap device 900 is similar to that of the device 1. However, in this case there is a backcheck valve 950 of similar construction to the valve 10 mounted within the opening 918 of the outlet conduit 917. This helps to ensure that the device 900 may be used in any desired orientation with inlet flow through the inlet port 904, with prevention of back-siphoning.

### Discussion on Methods of Use of the Devices

Nurses must prime any device as part of an IV giving set to reduce the risk of air entering the patient's vasculature and causing an air embolism. The device (300) is an open in-line filter that has a back-check valve (310) attached to the body, located over hydrophobic membranes that are intended to release air to the atmosphere. The backcheck valve is comprised of a pair of flexible walls that contact to form a seal, is normally closed and opens unidirectionally under a defined pressure to allow fluid or air to pass through in one direction only, then returns to its normally closed position to ensure a seal is formed. Environmental air or fluids cannot open the valve in the other direction; thus it is closed in one direction of flow.

The device (600) features the back-check valve (10) at the outlet port (605) of the device to ensure that flow can only be directed through the device in one direction, from the reservoir towards the patient. In use, if the device were raised higher than the patient's mid-axillary or IV site level, the flow of the device in the reverse direction would be stopped by the back-check valve, thus protecting the patient from harm and maintaining the prime in the device. To prevent the loss of prime in the device a cap (100, 102) that is selectively sealable can be used to optionally release air from the device, the device operating as a closed system when the cap is in the closed position. The cap in an open position enables initial priming of the device to occur by allowing expulsion of air through its open channel during the priming process.

During use, the release of gases from drugs, such as chemotherapy, blood or antibiotics, can lead to harm due to exposure in the workplace. It is therefore preferred that these gases are retained within the closed system, leading to a new subset of IV infusion devices known as Closed System devices, with Closed System Transfer Devices (CSTD) used mainly in pharmacy compounding and Closed System Administration Devices (CSAD) used mainly at the hospital bedside. To further prevent exposure of the infusion fluids, a closed system air trap device (500) can be attached to the open in-line filter (700) to act as reservoir for air that is released from the fluid via its hydrophobic filter (335). The device 500 is similarly intended to trap air as the device (302) when it is attached to the upper surface of an in-line filter device. The closed air trap device (300) may use back-check valves (310) and a shell (302) with a selectively sealable cap (320) to improve the safety and performance of the device (301). A shell may be broadly formed from any rigid, semi-rigid or flexible material in any number of designs or shapes and drawings supplied here are indicative only. It is feasible that this device, or any feature of this device, may improve the safety and performance of any type of filter when used for delivery of IV therapy.

In some examples, the device can be a component device that is attached to the surface of an inline open filter device so as to make it a closed filter device, the combination of component devices forming a selectively openable or fully sealed closed filter device that is intended for use removing air or particles as part of a closed system during medical infusion.

The following are some examples of advantages which arise form the invention, in which a backcheck valve as described avoids the filter being vented:

### Back Siphoning

When a vented filter is positioned below the patient heart level, if the flow stops, the vent on the filter will cause a flow of fluid from the patient to the vent. This has been observed where particle filters filled with blood due to back siphoning.

### Bolus

If the filter is raised above the patient, a vent would cause the fluid to bolus into the patient rate.

### Leak and Burst Through of Filter Membrane

Filter membranes have a burst through pressure where fluid can escape to atmosphere.
This again is a safety risk to health care providers when hazardous drugs are used.

Having a closed system filter of the invention solves each of the above problems.

Components shown as part on any embodiment may be applied to use with devices of any other embodiment, and the drawings are merely examples of desired combinations of components.

1. A fluid filter device for blocking of gas and/or particles in a medical fluid line, the device comprising:
an inlet,
an outlet, and
a flow chamber between said inlet and outlet for flow in a flow direction distally from the inlet to the outlet, wherein the flow chamber comprises:
an active component for trapping gas and/or particles, and
a port with a backcheck valve to allow escape of gas from the chamber but not inflow of gas into the chamber.

2. A device as claimed in claim 1, wherein the flow chamber comprises a gas trap, with the outlet configured for flow of only liquid with gas being trapped in the chamber either temporarily or permanently.

3. A device as claimed in claim 2, comprising a priming port for outflow of air from the flow chamber during priming and sealing to close the chamber for use.

4. A device as claimed in claim 3, wherein the backcheck valve is part of the priming port.

5. A device as claimed in claim 3, wherein there is a backcheck valve which is separate from the priming port.

6. A device as claimed in claim 5, wherein the backcheck valve comprises a pair of leaflets which are normally converged together and separate under air flow pressure from within the chamber.

7. A device as claimed in claim 1, wherein the flow chamber houses a particle filter.

8. A device as claimed in claim 7, wherein the flow chamber comprises a port with a backcheck valve proximally of the particle filter.

9. A device as claimed in claim 7, wherein the flow chamber comprises a port with a backcheck valve located adjacent the particle filter to accept outflow of gas laterally from the particle filter and the flow direction.

10. A device as claimed claim 1, wherein the device comprises an outer chamber for receiving gas which has flowed out through the port.

11. A device as claimed in claim 10, wherein the outer chamber is in fluid communication with a plurality of ports on the flow chamber.

12. A device as claimed in claim 1, wherein the device comprises a pressure regulator port (500), comprising a backcheck valve linked with a volume formed by a flexible diaphragm (503).

13. A device as claimed in claim 12, wherein the diaphragm forms a volume to trap gas which flows through the port, thereby preventing any toxic gases from escaping to the surrounding environment.

14. A device as claimed in claim 1, wherein the port comprises a mesh to retard outflow of liquid.

15. A device as claimed in claim 14, wherein the mesh is hydrophobic.

16. A device as claimed in claim 1, comprising a backcheck valve in the outlet.

17. A device as claimed in claim 1, comprising a gas trap and a priming port including a backcheck valve for permitting only outflow of gas.

18. A device as claimed in claim 1, comprising a port with a closure such as a cap, and a backcheck valve included in said port.

19. A medical intravenous giving set comprising a fluid supply, a supply line, and the device of claim 1 connected in the line to trap gas to prevent it from flowing towards a patient.
